(19) United States
(12) Patent Application Publication
(10) Pub. No.: US 2005/0244396 A1
(43) Pub. Date: Nov. 3, 2005

(54) Application of a non-toxic organic enzyme formulation and process for reducing fungi-caused decay on fruits and vegetables

(76) Inventor: Stephen J. Gorton, Rowlett, TX (US)

Correspondence Address: David S. Taylor, Esq.;Berenato, White & Stavish, LLC, Suite 240, 6550 Rock Spring Drive, Bethesda, MD, 20817, US

(21) Appl. No.: 11/179,630

(22) Filed: Jul. 13, 2005

Related U.S. Application Data
(63) Continuation of application No. 10/728,417, filed on Dec. 8, 2003.

(51) Int. Cl. A61K 38/54

(52) U.S. Cl. 424/94.2

(57) ABSTRACT

An enzyme formulation is provided for preventing fungi and their proteins from forming on fruits and vegetables, and for extending the shelf life of the fruits and vegetables from field (pre-harvest) to various post-harvest points, such as packing houses, processing operations, distribution points, shippers, retailers and consumers. The enzyme formula is comprised of a combination of safe, plant and vegetable based non-toxic organic enzymes and catalysts, and works by degrading and neutralizing the offending fungi, mycelium, spores and proteins on contact. The enzyme formulation is applied by identified methods to include standard spray applications, atomization/fumigation, dipping, or soaking and drenching.

### CROSS REFERENCE TO RELATED APPLICATION

This is a continuation of U.S. patent application Ser. No. 10/728,417 filed on Dec. 8, 2003, the complete disclosure of which is incorporated herein by reference.

### STATEMENT REGARDING FED SPONSORED R&D

(none)

### BACKGROUND OF THE INVENTION

The process of extending the pre-harvest and post-harvest life of fruits and vegetables is important (1) to increase overall crop yield, thereby expanding the general food supply; (2) to deliver more healthy, viable produce to the end-user without compromising consumer health through the consumption of pathogen-degraded fruits and vegetables; (3) to help maintain stable prices through abundant supply; and (4) to preserve commercial profitability. The invention has special importance in the processing and transportation of produce between field and market, as follows: climatic restrictions determine that fruits and vegetables can only be grown in certain parts of the world; after harvest, they must be shipped by carriers utilizing various modes of transportation including air, rail, truck and ocean vessels; they must travel through assorted and diverse climates; throughout this process they must be effectively protected from pathogen growth in order to arrive at final destination in viable condition; when not treated and protected in various ways while in the field and later, in transit, on store shelves, and finally after purchase by the consumer, the fruits and vegetables can and will be exposed to various damaging fungi and proteins on their outer surfaces, which will rapidly destroy the usefulness of the produce. The absolute certainty of degradation without effective pre-and post-harvest treatment, subjects growers, packers, processors, retailers and consumers to substantial losses. It would be a considerable advantage and help if these damaging influences could be eliminated or at least curtailed. Current methodologies fail to produce entirely satisfactory results. Therefore there is an immediate need for the technology of this invention, which has been developed specifically to address the crop protection/preservation concerns noted above.

Various laboratories throughout the country, including the Florida Department of Citrus, have identified the offending fungi of the above noted problems. The Florida Department of Citrus is especially interested in this endeavor because the State of Florida is a major producer of citrus fruits and other produce and ships the vast majority of the harvest domestically to all fifty states and exports to many offshore countries. As stated above, any losses related to growing, processing, storage and shipping, constitute significant financial burdens on growers, producers, packers, processors, shippers, distributors, retailers and consumers, as well as state tax revenues and a sizeable employment base, including direct and related jobs.

Awareness of fungal problems is well established in the scientific and industrial communities, and at the consumer level as well, including citrus specific pathogens, e.g., Diplodia natalnesis (stem-end rot); Colletotrichum gloeasporioides (anthracnose); Penicillium digitatum (green mold); Geotrichum candidum; and more.

Traditional remedies that have been used and are being used are chemical bleach washes and alkaline-based washes and other chemicals to include: (1) Sodium Ortho Phenyl Phenate (SOPP); (2) Imazalil; and (3) Thiabendazole (TBZ). Each of these is corrosive and may pose underlying human health hazards. Some of these remedies have been discredited as largely ineffective and unnecessarily costly and time consuming. They must be replaced by a more effective and safer technology, which is why the invention was formulated. An additional, albeit especially wasteful remedy, would be outright discard of large amounts of rotten fruits and vegetables that could have been saved with effective treatment. The innovation will be explained below.

### SUMMARY OF THE INVENTION

The offending fungi have been identified and include various spores, mycelium and related proteins. The enzyme-based invention was formulated specifically and exclusively to combat fungi, spores, mycelium and proteins. Safety and efficacy have been documented by laboratory studies, and by field applications and case studies across the country.

### DETAILED DESCRIPTION OF THE INVENTION

The unique, proprietary formula of the invention is composed of a combination of safe, non-toxic organic enzymes and catalysts using natural, plant-derived enzymes to break down polysaccharide cell walls, after which the enzymes enter, destroy and neutralize the protein content of the offending fungi, spores, mycelium and proteins on contact. This unique technology penetrates porous surfaces and leaves behind an effective surface inhibition, which protects the fruits and vegetables from fungal re-growth. The residual inhibition lasts up to one year on non-fruit and non-vegetable surface areas such as storage bins, harvesters, containers, packing crates, boxes and the like. The formulation continues to work until contiguous substrate sources have been eliminated. The invention used and applied, as described in the following methods and applications steps, is a non-toxic enzyme-based formulation that neutralizes fungi, mycelium, spores and proteins on contact.

The method of extending the pre-harvest (in the field) and post-harvest (processed) fruits and vegetables is to spray or surface coat the produce and related surface areas such as packing materials, boxes, crates, transportation equipment and the like, with the formula explained above. The treatment steps may involve four (4) or more methods.

1. Standard Spray Applications:

An application using a liquid spray involves coating the fruit or vegetable with the liquid enzyme formulation with particles greater than fifteen (15) microns in size.

2. Atomization Fumigation

The atomization fumigation application of the product involves misting the enzyme formulation with particles of less than fifteen (15) microns in size to treat the fruits and vegetables.

3. Dipping or Soaking

Dipping or soaking involves submersing the produce in the enzyme formulation, typically in a vat system or in a liquid holding tank. The dip process involves putting the fruits or vegetables into a container with a water base or a water base wax, and then removing the fruits or vegetables from the container.

4. Drenching

In the drenching method, the enzyme formulation is poured over the entire surface area of the produce.

In the above four (4) mentioned and discussed methods of application, it has been found that the shelf life of the fruits and vegetables has been substantially extended as the direct result. It is most likely that this significant improvement in post-harvest shelf life will deliver a greater volume of viable produce to market out of the same harvest; reduce chemical-related human health risks at all levels, including field worker, packer, processor, shipper, distributor, retailer and consumer; help maintain price stability with more effective and reliable crop protection/preservation methods thereby producing more reliable yield; preserve commercial profitability through the delivery to market of more abundant harvests; and increase state tax revenues.

The above mentioned processes allow for the enzyme-based formula to be applied both to pre-harvest and post-harvest crops to inhibit fungal spore germination and mycelium growth. This technology is also to be used to treat non-produce surface areas to limit and inhibit fungi growth on related surface areas to include processing and harvesting equipment, storage bins, containers, boxes, transportation containers, packing and shipping materials, and the like.

The unique, proprietary non-toxic organic enzyme-based formula of this invention protects plant workers against potential health risks, which risks they have been, and continue to be exposed to with the application of currently used chemical products.

1. A process for inhibiting onset of fungal invasion on surface areas of produce selected from fruits and vegetables during a period from pre-harvest through to post-harvest, comprising:
applying a formulation to the surface areas of the produce, the formulation comprising plant-derived, non-toxic organic enzymes and catalysts; and
enzymatically breaking down polysaccharide cell walls of fungi, mycelium, and spores on the surface areas of the produce and neutralizing protein of the fungi, mycelium, and spores.

2. The process of claim 1, wherein said applying comprises spraying the formulation as liquid particles greater than fifteen (15) microns to the surface areas of the produce.

3. The process of claim 1, wherein said applying comprises atomizing the formulation into liquid particle sizes of less than fifteen (15) microns.

4. The process of claim 1, wherein said applying comprises dipping of produce into a water-based liquid or water-based wax substance comprising the formulation.

5. The process of claim 1, wherein said applying comprises soaking of the produce into a water-based liquid or water-based wax substance comprising the formulation.

6. The process of claim 1, wherein said applying comprises drenching of the produce with a water-based liquid or water-based wax substance comprising the formulation.
